(19) United States
(12) Patent Application Publication
(10) Pub. No.: US 2026/0263654 A1
(43) Pub. Date: Sep. 10, 2026

(54) NOVEL HYDROGEL HAVING BIOCOMPATIBLE AND SELF-ADHESIVE PROPERTIES

(71) Applicant: INDUSTRY ACADEMIC COOPERATION FOUNDATION OF YEUNGNAM UNIVERSITY, Gyeongsan-si, Gyeongsangbuk-do (KR)

(72) Inventors: Sung Soo HAN, Daegu (KR); Suneetha MADURU, Gyeongsan-si, Gyeongsangbuk-do (KR); Madhusudana Rao KUMMARA, Gyeongsan-si, Gyeongsangbuk-do (KR)

(73) Assignee: INDUSTRY ACADEMIC COOPERATION FOUNDATION OF YEUNGNAM UNIVERSITY, Gyeongsan-si, Gyeongsangbuk-do (KR)

(21) Appl. No.: 19/165,575

(22) PCT Filed: Mar. 20, 2023
(86) PCT No.: PCT/KR2023/095012

§ 371 Date: Sep. 15, 2025

(30) Foreign Application Priority Data
Mar. 14, 2023 (KR) 10-2023-0033327

(51) Int. Cl. A61L 24/00; A61F 13/0203; A61L 15/58; A61L 24/04; C08L 33/26

(52) U.S. Cl. CPC A61L 24/0031; A61F 13/0213; A61L 15/58; A61L 24/0036; A61L 24/043; C08L 33/26

(57) ABSTRACT

The present invention relates to a novel hydrogel having biocompatible and self-adhesive properties. The hydrogel, which is prepared by combining dopamine and polyacrylamide with quaternized hydroxyethyl cellulose (QHEC), can be effectively used as a composition for wound healing or hemostasis as it is confirmed that the hydrogel has superior elasticity, tensile strength, and adhesion compared to existing hydrogels, promotes blood coagulation and moisture absorption, and exhibits antibacterial activity.

### TECHNICAL FIELD

The present disclosure relates to a novel hydrogel having biocompatibility and self-adhesiveness.

### BACKGROUND ART

Application of multifunctional properties of hydrogels in biomedical applications provides great potential as wound dressings and bioelectronic devices. In order to create these multifunctional hydrogels, various materials and strategic designs are required. The catechol structure found in mussel adhesive proteins has attracted much attention in development of artificial functional materials because it contributes to adhesion and cohesion formation of these proteins. Polydopamine, a polymer composed of catechol, possesses strong adhesion to various organic, inorganic, and substrate surfaces, as well as human skin, like mussel adhesive proteins. Additionally, it has excellent cell proliferation and cell activity with a structural similarity to mussel adhesive proteins. Therefore, this mussel-derived chemical substance sheds new light on the development of hydrogels with excellent cell activity and tissue adhesiveness.

Multifunctional hydrogels are being developed as wound dressings and flexible wearable devices in bioelectronics using various polymers and materials, and, while the hydrogel is applicable to other biomedical fields such as tissue engineering and drug delivery based on physicochemical and biological properties of hydrogels, integration of various properties, such as self-adhesiveness, repeatable adhesiveness, stretchability, high conductivity, excellent mechanical performance, and superior electrical stability, within a single hydrogel in bioelectronics is still a challenging task, thus further research is needed.

### DISCLOSURE OF THE INVENTION

### Technical Goals

An object of the present disclosure is to provide a biocompatible hydrogel (PDA-QHEC-PAM) including quaternized hydroxyethyl cellulose (QHEC), polydopamine (PDA), and polyacrylamide (PAM) as active ingredients.

Another object of the present disclosure is to provide a pharmaceutical composition for wound healing or hemostasis, including the hydrogel (PDA-QHEC-PAM) as an active ingredient.

Another object of the present disclosure is to provide a method of preparing the hydrogel (PDA-QHEC-PAM).

### Technical Solutions

To achieve the above objects, the present disclosure provides a biocompatible hydrogel (PDA-QHEC-PAM) including quaternized hydroxyethyl cellulose (QHEC), polydopamine (PDA), and polyacrylamide (PAM) as active ingredients.

In addition, the present disclosure provides a pharmaceutical composition for wound healing or hemostasis, including the hydrogel (PDA-QHEC-PAM) as an active ingredient.

In addition, the present disclosure provides a method of preparing a biocompatible hydrogel (PDA-QHEC-PAM) including: mixing quaternized hydroxyethyl cellulose (QHEC) and dopamine (DA) and solubilizing the mixture (first step); and mixing the mixture with acrylamide (AM) and polymerizing the same (second step).

### Advantageous Effects

According to the present disclosure, it was found that, compared to existing hydrogels, a hydrogel prepared by combining dopamine and acrylamide (polyacrylamide) with quaternized hydroxyethyl cellulose (QHEC) has superior elasticity, tensile strength, and adhesion, promotes blood coagulation and water uptake, and exhibits an antibacterial activity, and thus it may be usefully utilized as a composition for wound healing or hemostasis.

### BRIEF DESCRIPTION OF DRAWINGS

FIG. 1 shows a schematic diagram of formation and bonding interactions of an adhesive hydrogel.

FIG. 2 shows a result of analyzing FTIR spectra of a hydrogel.

FIG. 3 is a graph showing a result of analyzing a gel fraction (%) of a hydrogel according to a content of ammonium persulfate (APS).

FIGS. 4A to 4E show results of analyzing adhesion of a hydrogel on human skin, FIGS. 4F to 4L show results of analyzing adhesion of a hydrogel on adhesive skin (F), stainless steel (G), polytetrafluoroethylene (PTFE) (H), glass (I), rubber (J), plastic (K), and steel (L) with an adhesion force of 500 g, FIG. 4m shows a graph presenting a result of analyzing adhesion of a hydrogel to pig skin, and FIG. 4n is a schematic diagram of an adhesion mechanism of a hydrogel to tissues (N=3, *p<0.05).

FIG. 5 is an image of a hydrogel being peeled off from human skin to check adhesion of a hydrogel.

FIG. 6 is a graph showing a result of analyzing an adhesive strength of a hydrogel to various materials.

FIG. 7 shows a result of an XPS survey spectrum of a hydrogel.

FIG. 8 is a graph showing a result of analyzing the zeta potential of a hydrogel.

FIGS. 9A and 9B show results analyzing N1s XPS spectra for each type of hydrogel, FIG. 9C shows a storage modulus (G′) of a hydrogel, FIG. 9D shows a loss factor (tan δ), FIG. 9E shows a tensile stress-strain curve of a hydrogel, and FIG. 9f is a graph showing a result of analyzing DMA compression of a hydrogel.

FIG. 10A shows results of analyzing a self-healing ability of two cut hydrogels, FIG. 10B shows results of analyzing a self-healing ability of two uncut hydrogels, FIG. 10C shows a result of analyzing a strain amplitude rheology (amplitude 1-1500%; frequency (1 Hz)) of a hydrogel, FIG. 10D shows a result of analyzing rheology of a hydrogel at two different strains (1% and 1500%; frequency (1 Hz)) with continuous strain sweep time cycles, and FIG. 10E is a stress-strain curve graph of a hydrogel before and after self-healing.

FIGS. 11A to 11C are low-magnification scanning electron microscope (SEM) images of each type of hydrogel, FIGS. 11A-1 to 11C-1 are high-magnification scanning electron microscope (SEM) images of each type of hydrogel, FIGS. 11D to 11F show water contact angles of each type of hydrogel, and FIG. 11G shows an average value of the water contact angle.

FIGS. 12A and 12B are graphs showing results of analyzing a water uptake capacity (swelling) and biodegradation of a hydrogel, respectively.

FIGS. 13A and 13B show results of analyzing cell proliferation of skin fibroblasts and keratinocytes via MTT assay, respectively, FIG. 13C shows fluorescent images of skin fibroblasts and keratinocytes cultured with hydrogel (scale bar: 200 μm) (green indicates live cells and red indicates dead cells) (N=3, *p<0.05).

FIGS. 14A and 14B are scanning electron microscopy (SEM) images of skin fibroblasts and keratinocytes attached to a hydrogel, respectively.

FIGS. 15A and 15B show results of analyzing blood coagulation time and hemolysis rate (%) of a hydrogel, respectively, FIG. 15C shows scanning electron microscope (SEM) images of platelet attachment to a hydrogel, FIG. 15D shows a result of analyzing a bacterial reduction rate (%) for E. coli and Staphylococcus aureus treated with a hydrogel, and FIG. 15E shows a schematic diagram of a multifunctional hydrogel (N=3, *p<0.05).

FIGS. 16A and 16B are images of experiments for analyzing blood coagulation and hemolysis of a hydrogel, respectively.

FIG. 17 shows a result of analyzing an antibacterial activity of a hydrogel via a disc diffusion method.

### BEST MODE FOR CARRYING OUT THE INVENTION

Hereinafter, the present disclosure will be described in more detail.

The present disclosure provides a biocompatible hydrogel (PDA-QHEC-PAM) including quaternized hydroxyethyl cellulose (QHEC), polydopamine (PDA), and polyacrylamide (PAM) as active ingredients.

The hydrogel may further include N,N′-methylenebis(acrylamide) (BIS), and the N,N′-methylenebis(acrylamide) may be used as a cross-linking agent.

In addition, the hydrogel may be in a form cross-linked by N,N′-methylenebis(acrylamide), with QHEC serving as a polymer and polydopamine and polyacrylamide serving as monomers.

Additionally, the hydrogel may have one or more properties selected from the group consisting of self-adhesiveness, porosity, biodegradation, and hydrophilicity, but is not limited thereto.

Additionally, the hydrogel may promote blood coagulation or water uptake.

Additionally, the hydrogel may exhibit an antibacterial activity against Escherichia coli or Staphylococcus aureus, but is not limited thereto.

In addition, the present disclosure provides a pharmaceutical composition for wound healing or hemostasis, including the hydrogel (PDA-QHEC-PAM) as an active ingredient.

The pharmaceutical composition may be in the form of a topical preparation.

In other embodiments of the present disclosure, the pharmaceutical composition may further include one or more additives selected from the group consisting of appropriate carriers, excipients, disintegrators, sweeteners, coating agents, leavening agents, lubricants, glydents, flavoring agents, antioxidants, buffers, bacteriostatic agents, diluents, dispersants, surfactants, binders, and lubricants that are commonly used in the manufacture of pharmaceutical compositions.

The pharmaceutical composition according to the present disclosure may be applied in any formulation, and more specifically, it may be used by being formulated as an oral dosage form, a topical preparation, a suppository, and a parenteral dosage form of a sterile injection solution according to a conventional method, and preferably, it may be a topical preparation.

The dosage of the active ingredient according to the present disclosure may vary depending on the condition and body weight of a subject, the type and severity of a disease, the form of the drug, and the route and duration of administration and may be appropriately selected by those skilled in the art, and the daily dose may be 0.01 mg/kg to 200 mg/kg, preferably 0.1 mg/kg to 200 mg/kg, and more preferably 0.1 mg/kg to 100 mg/kg. The administration may be conducted once a day or divided into several doses, but the scope of the present disclosure is not limited thereby.

In addition, the present disclosure provides a method of preparing a biocompatible hydrogel (PDA-QHEC-PAM), including mixing quaternized hydroxyethyl cellulose (QHEC) and dopamine (DA) and solubilizing the mixture (first step), and mixing the mixture with acrylamide (AM) and polymerizing the same (second step).

The first step may further include mixing ammonium persulfate (APS) with QHEC.

The second step may further include mixing N,N′-methylenebis(acrylamide) (BIS), ammonium persulfate (APS), and N,N,N′,N′-tetramethylethylenediamine (TEMED) with the mixture.

Additionally, the second step may include polymerizing at 50 to 70° C. for 1 to 4 hours.

### MODES FOR CARRYING OUT THE INVENTION

Hereinafter, the present disclosure will be described in more detail through examples to help understanding of the present disclosure. However, examples below are merely intended to illustrate the present disclosure, and the scope of the present disclosure is not limited to the following examples. Examples of the present disclosure are provided to more completely explain the present disclosure to those skilled in the art.

### [Example 1] Experimental Preparation

Quaternized hydroxyethyl cellulose (QHEC), dopamine (hereinafter referred to as DA), N,N′-methylenebis(acrylamide) (hereinafter referred to as BIS), N,N,N′,N′-tetramethylethylenediamine (hereinafter referred to as TEMED), and ammonium persulfate (hereinafter referred to as APS) were purchased from Sigma-Aldrich, and sodium hydroxide (NaOH) and acrylamide (hereinafter referred to as AM) used herein were purchased from Dae-Jung Chemical Co., Korea. Double distilled water (hereinafter referred to as DDW) was used in all experiments.

### [Experimental Example 1] Preparation of a PDA-QHEC-PAM Hydrogel

Polydopamine-quaternized hydroxyethyl cellulose-polyacrylamide (PDA-QHEC-PAM) hydrogel was prepared via simple free radical polymerization using QHEC as a polymer, DA and AM as monomers, and BIS as a cross-linking agent. After solubilizing different amounts of DA in 2.5% QHEC, 50 mg of APS was added to promote pre-polymerization of DA in the QHEC solution. Then, AM, BIS, APS, and TEMED were added sequentially, and the reaction mixture was polymerized at 60° C. for 3 hours. TEMED was used to catalyze formation of free radicals resulting from the added APS to promote the polymer formation of AM and BIS. The formed hydrogel sample was placed in DDW for two days to remove unreacted monomers and cross-linking agent. Finally, the hydrogel samples were frozen at −80° C. for additional analysis. As shown in Table 1, various hydrogel samples were prepared depending on the weight ratio of DA to AM (hereinafter referred to as DA/AM).

TABLE 1

| | | 0.2PDA- | 0.4PDA- |
|---|---|---|---|
| Hydrogel | QHEC-PAM | QHEC-PAM | QHEC-PAM |
| DA/Am (Wt %) | — | 0.2 | 0.4 |
| DA (mg) | — | 4 | 8 |
| QHEC (g) | 0.25 | 0.25 | 0.25 |
| AM (g) | 2 | 2 | 2 |
| BIS (mg) | 5 | 5 | 5 |
| APS (mg) | 50 | 200 | 200 |
| TEMED (μL) | 10 | 10 | 10 |

### [Experimental Example 2] Characteristics of PDA-QHEC-PAM Hydrogel

### 2-1. Characterization of the Hydrogel

The surface morphology of the hydrogel sample prepared was observed using a scanning electron microscope (SEM; Hitachi S-4800). Interactions between functional groups on the hydrogel samples were analyzed by X-ray photoelectron spectroscopy (XPS-ESCALAB 250) using a monochromatic source at Al Kα and hν=1486.6 eV with a spot size of 200 μm. The rheological behavior of the hydrogel samples was characterized using an Anton Paar-Physica MCR301 Rheometer (PP25 parallel-plate geometry). For frequency sweep tests, hydrogels were loaded and analyzed in the range of 1-10 Hz at 25° C. with a constant strain (1%). Strain amplitude tests were carried out on hydrogels in the range of 0.1-1500% at a fixed frequency (1 Hz). The compressive properties of the hydrogel samples were analyzed by dynamic mechanical analysis (DMA-Q800, TA Instrument). Hydrogel (cylindrical, 10 mm in height×10 mm in diameter) was applied in compression mode of 3.0 N min⁻¹ at 25° C. (preload: 0.01 N). The tensile stress-strain curves of the hydrogels were characterized using a mechanical tensile tester (MCT 2150, A&D Co., Ltd., Japan). The length, thickness, and width of the specimen were 20 mm, 10 mm, and 10 mm, respectively, and the loading rate was set to 100 mm·min⁻¹. The zeta potential of hydrogels (pure QHEC, 0.2PDA-QHEC-PAM, and 0.4PDA-QHEC-PAM) was analyzed using a Malvern Zetasizer Nano ZS90 (Malvern instrument at 25° C. with disposable cells). The surface wettability of the hydrogel was analyzed by the sessile water drop method (physics instruments-OCA20) at 25° C. Before contact angle measurement, the freeze-dried hydrogel was cut into small pieces and attached to a glass slide. Finally, water (10 μL) was dropped onto a hydrogel support, and an image was captured after 2 seconds. The contact angles of all hydrogel supports were measured in triplicate (n=3). The self-healing performance of the hydrogel samples was evaluated using a tensile tester (MCT 2150, A&D Co., Ltd., Japan) and a dynamic rheometer (Rheometer Physica MCR301; Anton Paar). The hydrogel sample was cut in half, and the two broken halves were brought into physical contact. After 2 hours, two hydrogels were self-healed, and the healed surface specimens of the healed hydrogels were characterized.

### 2-2. Analysis of Porosity

To determine the porosity of the hydrogel, a known weight of lyophilized hydrogel (W₁) was saturated by being immersed in a known volume of anhydrous ethanol in a sealed bottle for 48 hours. Afterwards, the hydrogel sample was removed from the bottle, and excess ethanol was wiped off from the hydrogel surface using a wiper. Finally, the weight of the hydrogel sample (W₂) was measured. The hydrogel porosity was calculated using the following Equation 1 (V=hydrogel volume, ρ=density of anhydrous ethanol).

$$Porosity ( \% ) = \frac{{W}_{2} - {W}_{1}}{ρ V} \times 100 \qquad Equation 1$$

### 2-3. Analysis of a Water Uptake Capacity and Biodegradability

To determine the water uptake capacity of the hydrogel, the hydrogel sample was cut into small pieces, the dry weight (W_{d}) was first measured, and the sample pieces were immersed in phosphate-buffered saline (PBS) solution (10 mL), followed by culture at 37° C. for different time intervals (1, 3, 5, 7, 14, and 21 days). The PBS solution was replaced every other day. Afterwards, the sample piece was removed from the PBS solution, wiped with a wiper to remove excess water and swollen hydrogel sample adhered onto the hydrogel, and then the weight was measured by the swelling weight (Wₛ) of the hydrogel. The water uptake capacity (%) of the hydrogel sample was calculated using the following Equation 2.

$$Water uptake capacity ( \% ) = \frac{{W}_{s} - {W}_{d}}{{W}_{d}} \times 100 \qquad Equation 2$$

In addition, to identify the biodegradability of the hydrogel, the swollen hydrogel sample was washed with DDW and freeze-dried at −80° C., and finally the weight (W_{f}) of the freeze-dried hydrogel sample was measured. The weight loss (%) was calculated using the following Equation 3 to measure the biodegradability of the hydrogel.

$$Weight loss ( \% ) = \frac{{W}_{d} - {W}_{f}}{{W}_{d}} \times 100 \qquad Equation 3$$

### 2-4. Analysis on the Adhesion of Hydrogels to Pig Skin

To determine the adhesion of the hydrogel to pig skin, a tensile tester (MCT 2150, A & A&D Co., Ltd., Japan) was used (N=3 for each experiment). The hydrogel was fixed between two pig skin specimens with a bonding area of 25×20 mm, and the adhesion was measured by dividing the maximum load (N) by a bonding area (A).

### 2-5. In-Vitro Cell Studies

Fibroblasts and keratinocytes purchased from ATCC were seeded in Dulbecco's modified Eagle's medium (DMEM) supplemented with 10% fetal bovine serum (FBS) and 1% antibiotic (penicillin-streptomycin; PS) solution, followed by culture in a humidified chamber at 37° C. in the presence of 5% CO₂.

For the live/dead assay, the two types of cells with a density of 5×10⁴ were cultured in a 24-well plate with hydrogel in a humidified incubator for 3 and 7 days, respectively, followed by measurement. Cells were treated with live/dead assay solution (500 μL) and cultured at room temperature for 30 minutes. Fluorescent images of live/dead cells present in the hydrogel were obtained using a Nikon Eclipse Ti-fluorescence microscope.

For cell adhesion, the two types of cells (5×10⁴ cm⁻²) were cultured in the hydrogel for different time intervals (3 days and 7 days), respectively. Cells were immobilized by adding 4% formaldehyde solution and cultured at room temperature for 10 minutes. The hydrogel-loaded cells were washed with DDW and then dehydrated with 30, 50, 70, and 100% ethanol-water mixtures. Cells attached to the hydrogel sample were observed with a field emission scanning electron microscope (hereinafter referred to as FE-SEM).

For proliferation, the two types of cells (5×10⁴ cm⁻²) were seeded on hydrogels fixed to 24-well plates and cultured for 1, 3, 7, and 14 days. After removing the medium at predetermined time intervals, 200 μL of MTT dye solution (5 mg/mL PBS solution) was added to each well. After 4 hours of culture, acidic isopropanol solution was added to form and dissolve formazan crystals, followed by additional culture for 30 minutes in a dark room at room temperature. The solution generated from each well was transferred to a 96-well plate, and the optical density value was measured at a wavelength of 570 nm using a microplate reader (Biotek Instrument, USA).

### 2-6. Analysis of the Blood Coagulation Time

Fresh porcine blood (Lotte Foods Co., Ltd. slaughterhouse) was collected in sodium citrate (3 mg/mL blood). A 300 μL of a sample of recalcified porcine blood was added to the hydrogels (QHEC-PAM, 0.2PDA-QHEC-PAM, and 0.4PDA-QHEC-PAM) mounted onto a 42-well plate. The wells were washed with saline solution at specific time intervals to remove uncoagulated blood components. A stable blood clot was formed at a specific time, measured as blood coagulation time (s).

### 2-7. Hemolysis Test and Platelet Adhesion

Citrate-treated porcine blood was centrifuged to separate red blood cells (RBCs) and platelet-rich plasma (PRP). 1 mL of concentrated red blood cell sample was diluted with 9 mL of saline solution. Then, the hemolytic activity of the hydrogel was analyzed. 100 mg of hydrogel samples (QHEC-PAM, 0.2PDA-QHEC-PAM, and 0.4PDA-QHEC-PAM), 100 μL of 0.1% Triton-X (positive control), and saline (negative control) were added to a conical tube. Next, 500 μL of diluted red blood cells were added and cultured at 37° C. for 1 hour. After culture, the sample was centrifuged at 3500 rpm for 10 minutes to obtain the supernatant. The collected supernatant was transferred to a 96-well plate, and the optical density (OD) was measured at 540 nm using a microplate reader (Epoch Microplate spectrophotometer, Biotek Instrument, USA). Hemolysis (%) was calculated using the following Equation 4.

$$Hemolysis ( \% ) = \frac{OD of hydrogel sample - OD of negative control}{OD of postitive control - OD of negative control} \times 100 \qquad Equation 4$$

For platelet adhesion tests, hydrogels (QHEC-PAM, 0.2PDA-QHEC-PAM, and 0.4PDA-QHEC-PAM) were immersed in PBS solution for 3 hours. Plasma (PRP) suspension was placed in a 24-well plate with a hydrogel plate and cultured at 37° C. for 1 hour. Platelets were fixed for 2 hours using 1% glutaraldehyde solution. Finally, the hydrogel samples were washed with DDW and dehydrated using a series of ethanol-water (30%, 50%, 70%, and 100% ethanol). Platelets adhered to the hydrogel samples were analyzed using FE-SEM (Hitachi S-4800).

### 2-8. Analysis of the Antibacterial Activity

To identify the antibacterial activity of the hydrogel, zone inhibition and colony forming unit assays were performed using Gram-negative (E. coli) and Gram-positive (Staphylococcus aureus) bacteria. After 100 μL of bacterial suspension was uniformly spread on an agar plate using a sterile glass spreader, a hydrogel with a diameter of 6 mm was applied to the bacterial agar plate. Afterwards, the inoculated agar plates were cultured in an incubator at 37° C. for 12 hours, and the antibacterial activity was measured by the zone of inhibition (mm) appearing around the hydrogel plate (disc). The CFU method was used to evaluate the hydrogels (QHEC-PAM, 0.2PDA-QHEC-PAM, and 0.4PDA-QHEC-PAM) using Escherichia coli and Staphylococcus aureus. Bacteria were inoculated into Luria-Bertani (LB) growth medium at 37° C. at 150 rpm for 24 hours. Colonies were removed using an inoculation ring in 50 mL of nutrient medium at 37° C. for 12 hours. A 4 mL of medium sample containing bacteria at a concentration of 10⁶ CFU/mL was cultured with the hydrogel (size: 15×15×1 mm). A bacterial solution without hydrogel was used as a control. Finally, the glass vials were cultured at 37° C. and 150 rpm for 24 hours. For CFU testing, 200 μL of bacterial solution was evenly spread on an agar plate. Bacterial colony forming units were counted after an additional 24 hours of culture on agar plates at 37° C.

### [Experimental Example 3] Statistical Analysis

All data are presented as mean±standard deviation (SD) (N=3). Statistical differences were determined using one-way ANOVA, and p<0.05 was considered significant.

### [Example 1] Characterization of the PDA-QHEC-PAM Hydrogel

### 1-1. Analysis of Gel Fraction

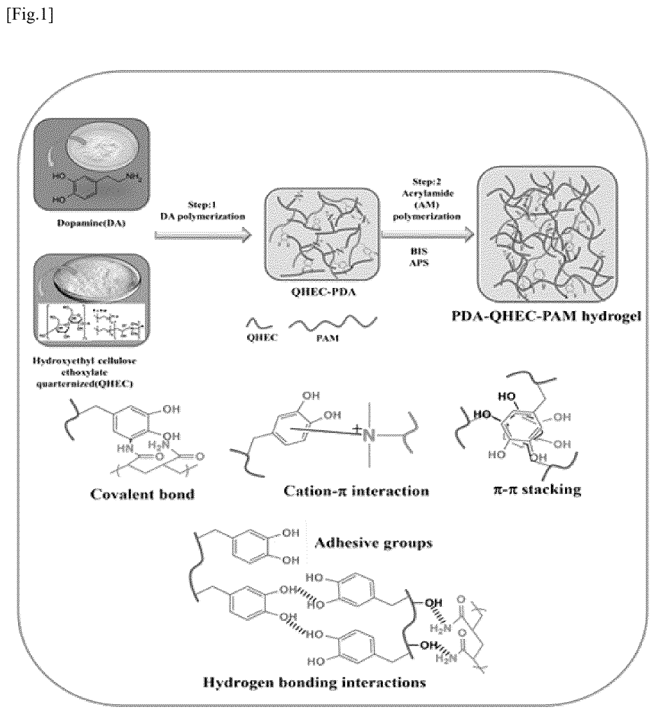

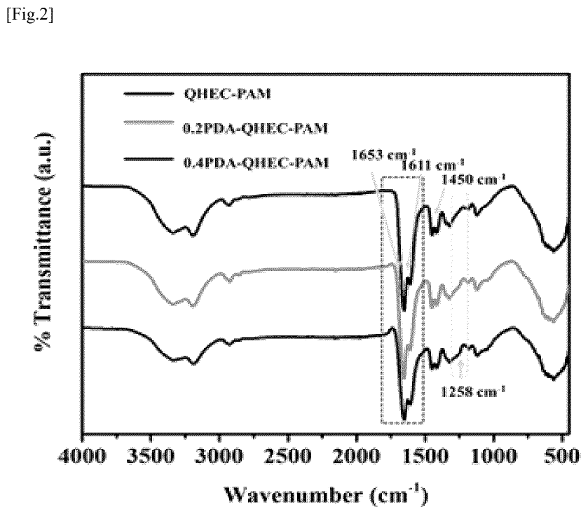

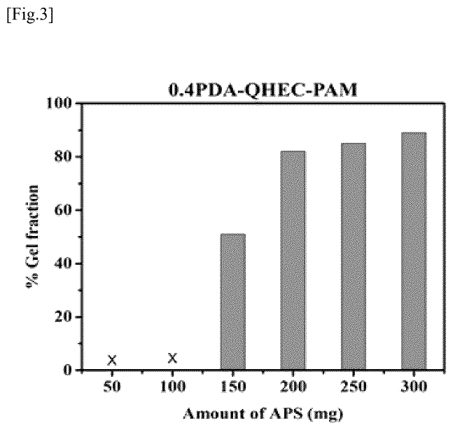

As shown in FIG. 1, the PDA-QHEC-PAM hydrogel was prepared. First, DA was polymerized in QHEC solution in the presence of APS. Most studies involved the use of alkali-induced polymerization by DA and free radical polymerization of monomers. In this approach, APS was used directly for both DA and AM polymerizations to avoid the original functionality of QHEC. In the second step, the remaining APS was participated in the polymerization of AM using BIS as a cross-linking agent at 60° C. The amount of DA affected the physicochemical properties of the hydrogel, showing that polymerization of AM was not feasible at higher amounts of DA (>0.4 wt % DA/AM) since APS molecules could be involved in the formation of PDA. Therefore, it was found that the remaining APS was not sufficient to initiate polymerization of AM monomers. As shown in FIG. 2, PDA was shown to affect a polymerization efficiency of the PDA-QHEC-PAM hydrogel. Based on the gel fraction (%) calculation, as a result of analyzing the effect of APS on the formation of 0.4PDA-QHEC-PAM hydrogel, the hydrogel was not formed at less than 100 mg of APS. As shown in FIG. 3, when 150 mg of APS was used, the gel fraction of the 0.4PDA-QHEC-PAM hydrogel was very low, whereas the gel fraction reached 82% when 200 mg of APS was used. From the results, it was determined that the gel fraction increased as the amount of APS increased. Therefore, in this study, the APS used in manufacturing hydrogel was fixed to 200 mg. Additionally, as shown in FIG. 1, the FTIR spectrum showed that, in the hydrogel structure, the PDA chains act as cross-linkers to connect with the QHEC and PAM chains by forming reversible non-covalent bonds such as hydrogen bonds, 71-71 stacking, and cation-π interactions.

### 1-2. Analysis of Adhesion

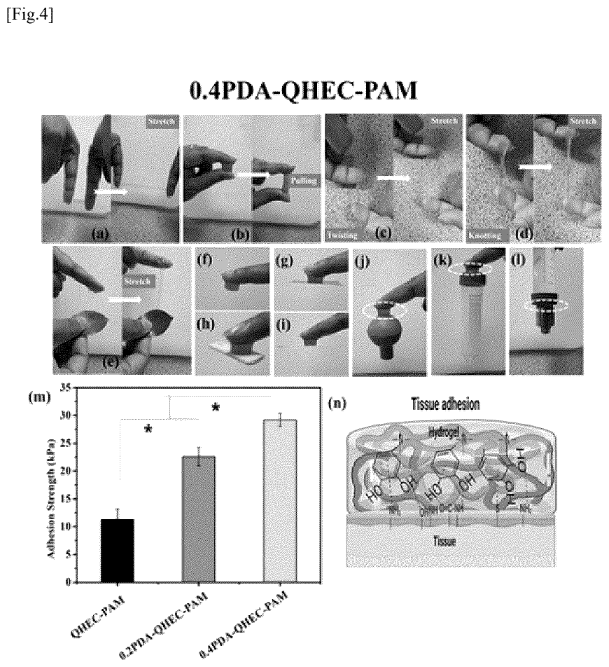

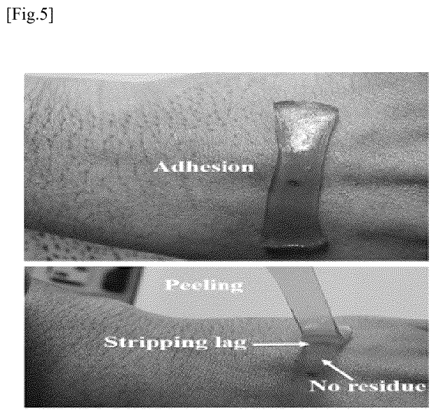

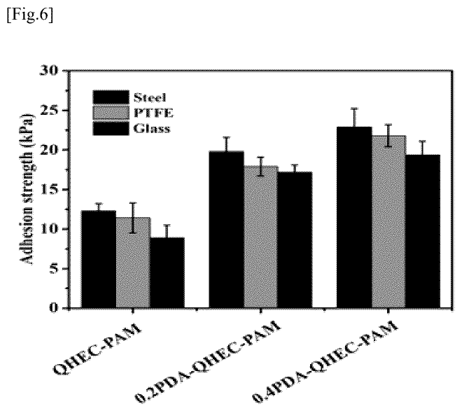

To analyze the adhesion of the hydrogel, as shown in FIGS. 4a to 4e, the 0.4PDA-QHEC-PAM hydrogel was stretched, pulled, twisted, and knotted from its original state with a finger, and it was found that the hydrogel had excellent adhesion. In addition, as shown in FIGS. 4f-4i, the 0.4PDA-QHEC-PAM hydrogel exhibited excellent adhesion to a wide range of substrates such as skin, stainless steel, PTFE, glass, rubber, plastic, and iron with an adhesion force of 500 g, and peeling lag was observed when the hydrogel was peeled off from the skin surface as shown in FIG. 5. Additionally, the adhesion strengths of QHEC-PAM, 0.2PDA-QHEC-PAM, and 0.4PDA-QHEC-PAM hydrogels to pig skin were 11.3±1.9 kPa, 22.6±1.7 kPa, and 29.3±1.2 kPa, respectively. Specifically, as shown in FIGS. 4m and 4n, the 0.4PDA-QHEC-PAM hydrogel was found to have the best adhesive strength to pig skin among the three types of hydrogels due to the large number of free catechol groups and cationic groups in the hydrogel network. In addition, as shown in FIG. 6, it was found that the adhesive strength of the hydrogel to other substrates such as steel, PTFE, and glass was improved by increasing the amount of DA in the hydrogel.

### 1-3. Analysis of the Composition and Polymerization Efficiency

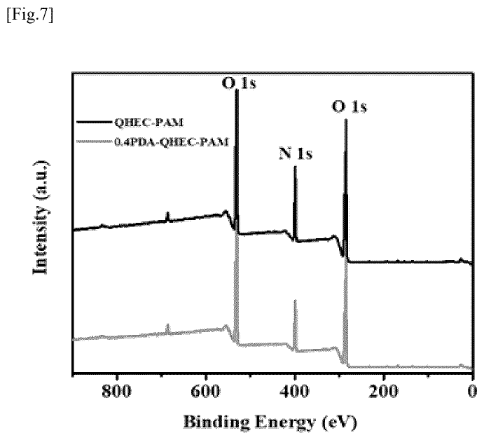

To evaluate elements in the constitutive functional group of the hydrogel, as a result of conducting X-ray photoelectron spectroscopy (XPS), as shown in FIG. 7, the QHEC-PAM hydrogel showed C 1s, N 1s, and O 1s peaks with atomic percentages of 61.21, 15.92, and 22.8, respectively, and the PDA-QHEC-PAM hydrogel showed C is, N is, and O is peaks with atomic percentages of 61.77, 14.09, and 24.14, respectively.

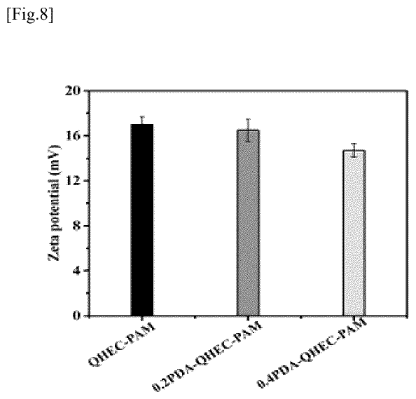

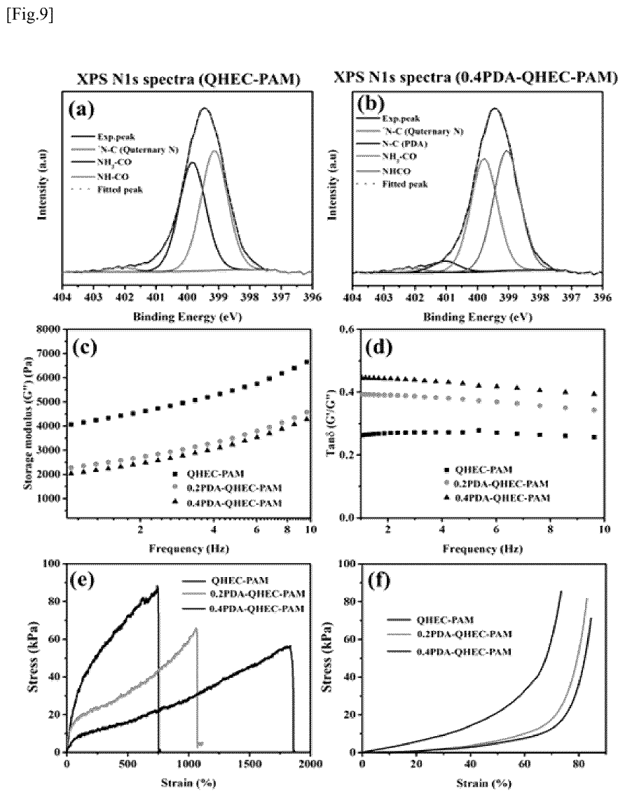

The polymerization efficiency of the hydrogel was identified through XPS spectra. To determine the positive charge on the hydrogel surface, as a result of screening the zeta potential of the hydrogels, as shown in FIG. 8, the QHEC-PAM, 0.2PDA-QHEC-PAM, and 0.4PDA-QHEC-PAM hydrogels exhibited positive zeta potential values of approximately 17.0±0.7, 16.5±1.1, and 14.7±0.6 eV, respectively. To determine the positive charge of the hydrogel, as a result of further analysis by performing deconvolution XPS spectrum of N is, as shown in FIG. 9a, the QHEC-PAM hydrogel exhibited a curve of three peak components with binding energies at 399.1, 400.1, and 402.1 eV, which were attributed to —NH, —NH₂ (amide) groups, and quaternary ammonium salt groups, respectively. Additionally, as shown in FIG. 9b, the 0.4PDA-QHEC-PAM hydrogel exhibited three binding energies at 399.1, 400.2, 401.1, and 402.1 eV from —NH, —NH₂ (amide), —NH(PDA), and quaternary ammonium salt groups, respectively. Additionally, it was found that the quaternary ammonium salt in the hydrogel network possesses a cationic charge, which is primarily responsible for the antibacterial properties in the hydrogel and enhances blood coagulation.

### 1-4. Analysis of Rheological and Mechanical Properties

As shown in FIGS. 9c and 9d, as a result of performing the frequency sweep by rheological measurements, an increase in the DA content increased the viscosity of the hydrogel to lead to a decrease in the storage modulus (G′) of the hydrogel, and the QHEC-PAM hydrogel showed superior elastic behavior compared to the 0.2PDA-QHEC-PAM and 0.4PDA-QHEC-PAM hydrogels. From the results, it was found that DA increases the viscosity of the hydrogel.

Although increased viscosity may limit the load-bearing properties, it was also discovered that viscosity is beneficial for the self-healing ability of hydrogels. As shown in FIG. 9e, the tensile stresses of the QHEC-PAM, 0.2PDA-QHEC-PAM, and 0.4PDA-QHEC-PAM hydrogels were shown to be 87.5, 64.7, and 55.7 kPa, respectively, with reduction in maximum tensile stress with the increase in the DA content, while the tensile strain increased as the DA content reached 756%, 1073%, and 1828%, respectively. The decrease in tensile strength and the increase in tensile strain by increasing DA content were attributed to the increase in viscosity of the hydrogel, which was supported by the rheological results.

As a result of analyzing the compression properties of the hydrogel through dynamic mechanical, as shown in FIG. 9f, the hydrogel showed good compression properties without being broken. Specifically, the compressive strength of the QHEC-PAM hydrogel was approximately 71.5 kPa, and the stiffness at 70% strain was 1685.0 N m⁻¹. Incorporation of PDA decreased the compressive strength and stiffness of 0.2PDA-QHEC-PAM and 0.4PDA-QHEC-PAM hydrogels to 53.6 and 36.3 kPa, and 1320.0 and 899.8 N m⁻¹ at 80% strain, respectively. Despite the decrease in compressive strength and stiffness, the hydrogel had excellent elasticity and resilience, making it desirable for application to wound dressing.

### 1-5. Analysis of Self-Healing Performance

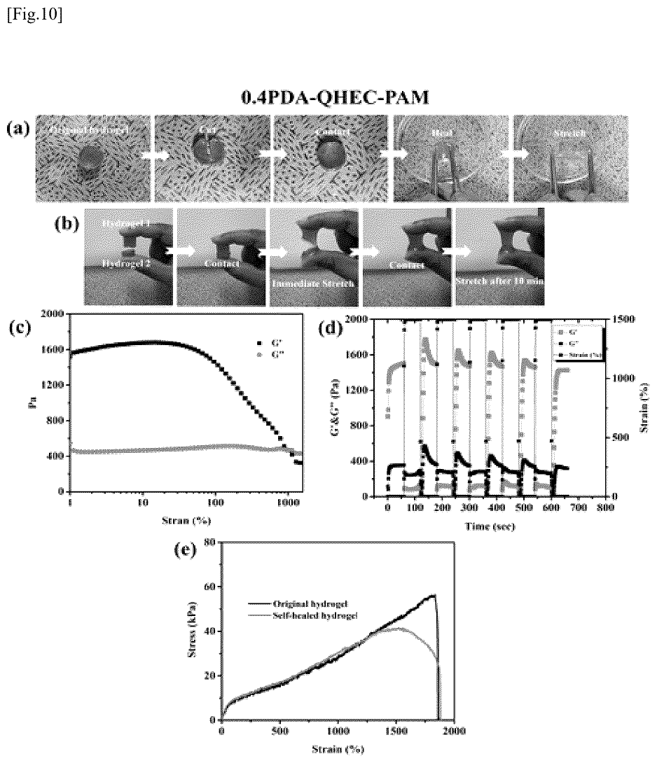

As shown in FIG. 10a, the 0.4PDA-QHEC-PAM hydrogel was cut into two pieces and then physically assembled. When stretching the hydrogel, no cut marks were recognized, which is thought to be because the newly cut hydrogel surface had many pendant catechol groups in the PDA and cationic moiety, resulting in strong self-healing ability. Additionally, when two uncut hydrogel discs were brought into physical contact (FIG. 10b) and the two discs were directly pulled, self-healing was not observed. When the PDA-PAM based hydrogel was in contact, self-healing did not take place for the two separated hydrogel discs. In contrast, after 2 hours, specific adhesion was observed between the two different hydrogel discs. The present study showed that the hydrogel self-healed after 10 minutes when two non-separated hydrogel discs were combined by physical contact due to the abundant cationic moieties, catechol groups, and other functional groups (—C═O, —CO—NH₂ and —CO—NH—) that can function as dynamic bonds for formation of multiple interactions such as H-bonding, −π, and cation π. The rapid self-healing ability was mainly attributed to the stabilization of electrostatic interactions between the polarizable π-electron cloud of PDA and the quaternary cation of QHEC.

The self-healing ability of the hydrogel was observed using dynamic rheology and tensile tests. Initially, strain amplitude tests for 1-1500% were performed on the 0.4PDA-QHEC-PAM hydrogel (FIG. 10c). As a result, the storage modulus was high under low strain conditions and stable up to 100%, and then it decreased further to lead to an increase in the strain (%). The intersection point of the storage modulus (G″) and the loss modulus (G′) at 1200% strain was attributed to the collapse of the hydrogel. Based on this experiment, as a result of conducting dynamic rheological experiments on two different strains (E. coli and Staphylococcus aureus) at predetermined time intervals (60 s) to recover the hydrogel (FIG. 10d), G′ of the hydrogel was shown to be stable and higher than G″ (G′>G″) at low strain (1%), presenting a solid gel state of the hydrogel. On the other hand, when the strain was suddenly switched to 1500% in the second time, G′ decreased drastically and G″ increased rapidly to a stable point (G″>G′), showing that the hydrogel was destroyed. In the third cycle, the strain was switched back to a low value (1%), and G′ and G″ were restored to their original states, i.e., G′>G″, indicating that the hydrogel returned to its original solid gel state. Dynamic self-healing behavior was observed for repetitive 6 cycles. Additionally, the tensile stress (39.6 kPa) of the healed sample (0.4PDA-QHEC-PAM hydrogel) was reduced by 23% compared to the initial hydrogel, showing good self-healing properties (FIG. 10e).

### 1-6. Analysis of the Microstructure and Wettability of Hydrogels

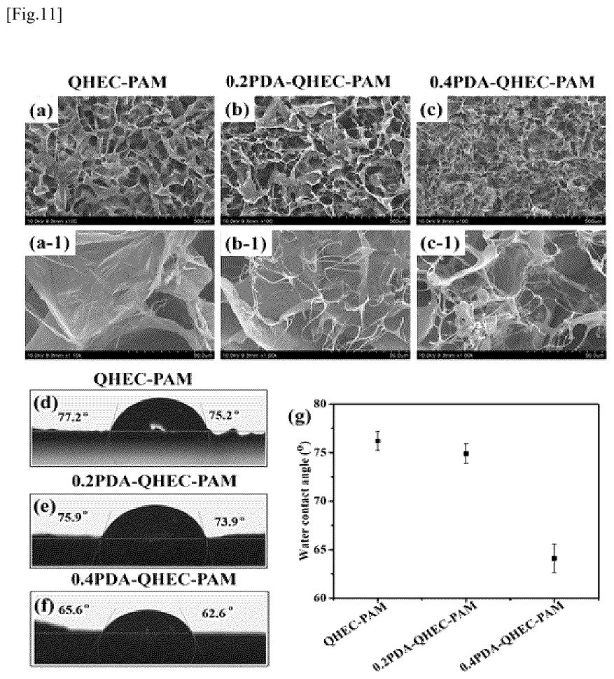

The morphology and microstructure of the freeze-dried hydrogel were analyzed using a scanning electron microscope (SEM). The hydrogel had a porous structure with a distinct shape (FIG. 11). The surface morphology of QHEC-PAM had a smooth surface (FIG. 11a-1), whereas the hydrogel containing PDA exhibited a microfibrous structure with a porous structure (FIGS. 11b-1 and 11c-1). In general, DA formed intermolecular interactions with various polymers, which resulted in the formation of interwoven microfibrillar structures. So far, the hydrogel morphology has shown that PDA microfibrils are formed with a porous structure. High-resolution SEM images of 0.2PDA-QHEC-PAM (FIG. 11b-1) and 0.4PDA-QHEC-PAM (FIG. 11c-1) hydrogels showed microfibrillar morphology linked to the QHEC-PAM hydrogel network (FIG. 11a-1). For QHEC-PAM, 0.2PDA-QHEC-PAM, and 0.4PDA-QHEC-PAM hydrogels, the average pore sizes were 123±17.0, 153±12.5, and 165±7.1, respectively. The results showed the maintenance of a porous structure with PDA microfibrils. The elevated PDA content increased the microfibrils of PDA-QHEC-PAM. Since PDA microfibrils are beneficial for cell function and hemostatic performance, the hydrogel was deemed useful as a wound dressing. Additionally, the porosity of the hydrogel was calculated using the solvent displacement method. For QHEC-PAM, 0.2PDA-QHEC-PAM, and 0.4PDA-QHEC-PAM hydrogels, the average porosities were found to be 84.3±2.4, 90.0±1.3, and 92.0±1.4%, respectively. Therefore, the increase in the PDA content in the hydrogel improved the porosity of the hydrogel.

To observe the hydrophilicity/hydrophobicity of the hydrogel, as a result of conducting WCA analysis to analyze the surface wettability, the range of WCA was 64° to 76°, indicating that the hydrogel was hydrophilic due to the abundance of hydrophilic functional groups in the polymer chain (FIGS. 11d-11g). The WCA of QHEC-PAM, 0.2PDA-QHEC-PAM, and 0.4PDA-QHEC-PAM hydrogels were 76.2±2.2°, 74.9±1.6°, and 64.1±2.1°, respectively. The WCA of the hydrogels decreased with the increase in the DA content, which was attributed to the increased hydrophilic functionality of the hydrogels due to the amine, hydroxyl, and carbonyl functional groups that are abundant in the QHEC, PAM, and PDA polymer chains. Therefore, it was determined that hydrogels may have the potential to absorb moisture to accelerate wound healing.

### 1-7. Analysis of the Water Uptake Capacity and Biodegradation

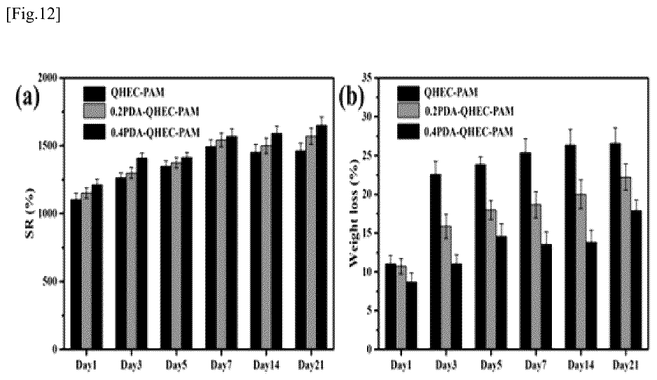

Swelling was observed on the hydrogel in PBS (FIG. 12a). The PDA-QHEC-PAM hydrogel showed better swelling capacity than the hydrogel without dopamine (QHEC-PAM), and the swelling ratio increased as the dopamine (DA) content increases over time. The swelling results were well consistent with the surface wettability outcome. Therefore, it was found that hydrogel is able to maintain the wound condition intact by transporting nutrients and amino acids to cells and promoting the uptake capacity of exudate from the wound, thereby retaining a large amount of moisture around the wound site and keeping the wound relatively dry.

Additionally, the combination of QHEC and PDA-PAM hydrogel showed biodegradation during contact with the wound (FIG. 12b). Hydrogel degradation was demonstrated using in vitro biodegradation of PDA-QHEC-PAM hydrogels by monitoring the weight loss as a function of immersion time. The initial weight (1 day of immersion) for QHEC-PAM, 0.2PDA-QHEC-PAM, and 0.4PDA-QHEC-PAM hydrogels decreased by 3.6%, 2.9%, and 1.8%, respectively. The weight loss increased as the immersion time increases. The maximum weight losses of QHEC-PAM, 0.2PDA-QHEC-PAM, and 0.4PDA-QHEC-PAM hydrogels during 21 days of immersion were 27.3%, 22.6%, and 17.1%, respectively. The weight loss for hydrogels containing large amounts of PDA showed strong interaction of QHEC with the amorphous polymer during hydrolysis of the hydrogel scaffold.

### 1-8. In Vitro Cell Studies

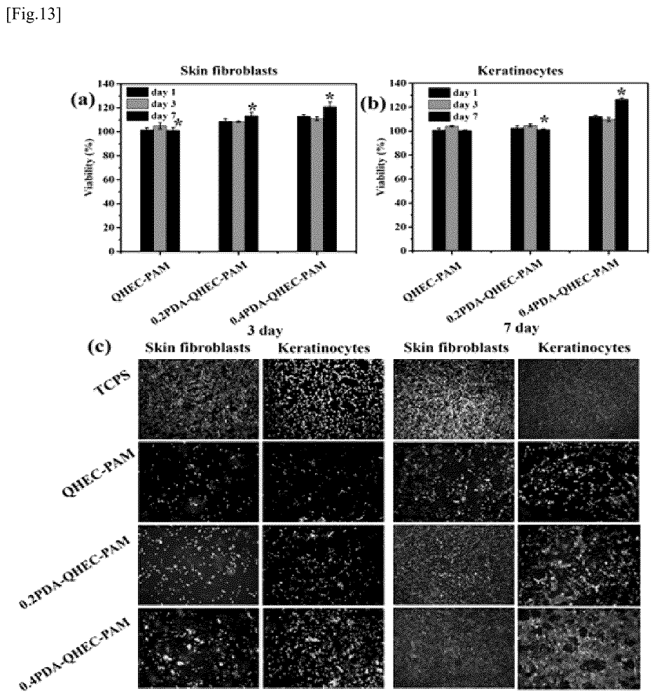

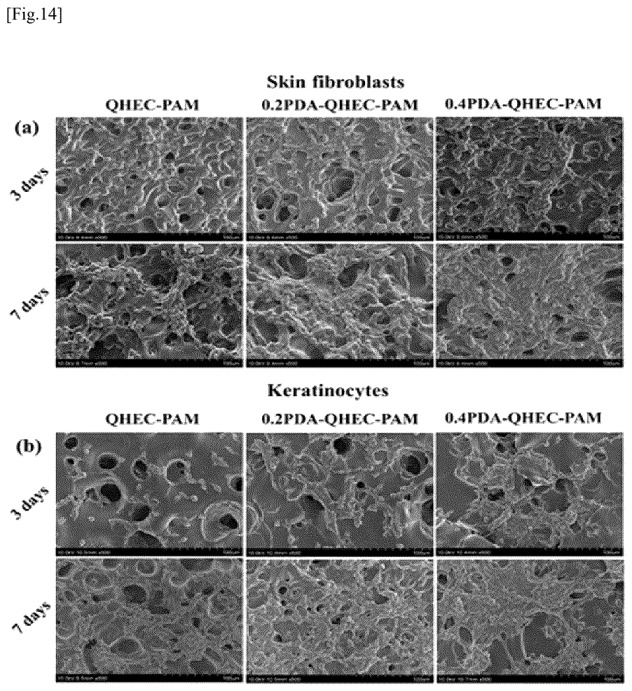

Two types of cells, dermal fibroblasts and corneal cells, were seeded in a hydrogel matrix to analyze cell adhesion and growth in the hydrogel. Results showed that the hydrogels were biocompatible when exposed to culture conditions for 7 days (FIGS. 13a and 13b). These results showed that the enhanced survival rates (%) of both cell types in the hydrogels were due to PDA, thereby promoting cell growth. Live/dead assay was performed on the cell viability of two cell types under 3-day and 7-day culture conditions in the hydrogel (FIG. 13c). These results showed that live cells (green, both cell types) were well distributed in the hydrogel with a negligible number of dead cells (red). Cells adhered and distributed in a higher degree onto the 0.4PDA-QHEC-PAM hydrogel than onto the QHEC-PAM and 0.2PDA-QHEC-PAM, additionally revealing that PDA plays an important role in cell growth and distribution throughout the hydrogel matrix. Additionally, as a result of analyzing the cell adhesion of the two cell types in the hydrogel by SEM, the morphology of the two cells (SEM) in the hydrogel showed appropriate cell adhesion and proliferation after 3 to 7 days of culture conditions, as shown in FIGS. 14a and 14b. In addition, due to the higher PDA content, cells were better attached and spread on the 0.4PDA-QHEC-PAM hydrogel compared to the QHEC-PAM and 0.2PDA-QHEC-PAM hydrogels. Overall, the adhesive hydrogels showed favorable cell proliferation, adhesion, and proliferation.

### 1-9. Analysis of Hemostatic Performance

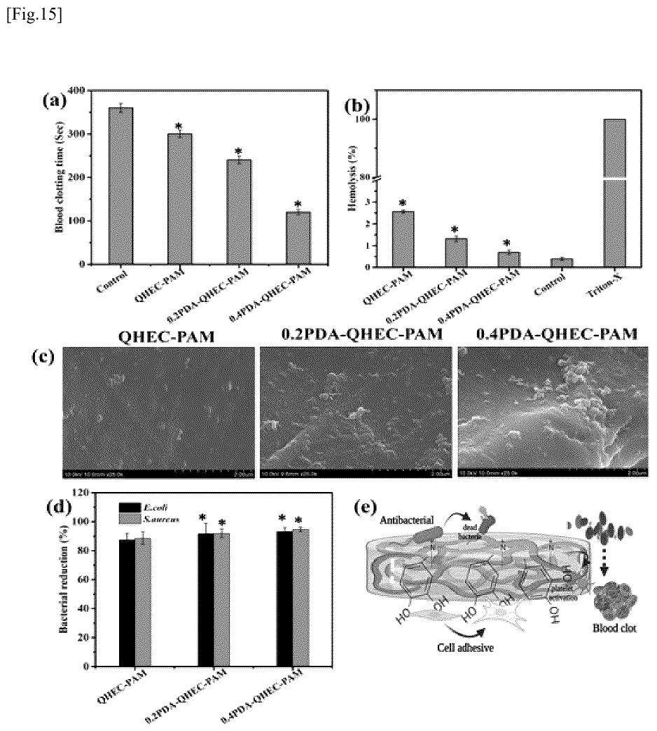

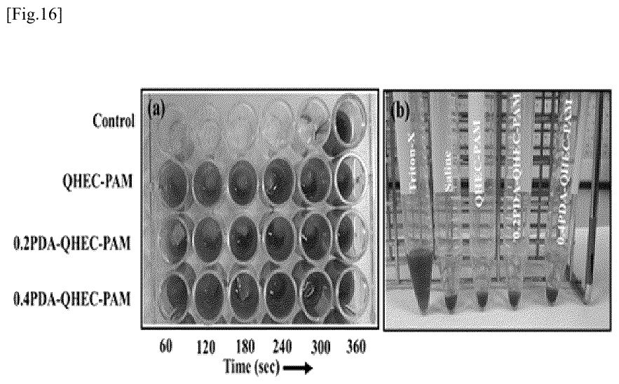

To determine the hemostatic performance of the hydrogel, the blood coagulation time was monitored. The combination of QHEC and PDA improved hemostatic performance. The blood coagulation times of QHEC-PAM, 0.2PDA-QHEC-PAM, and 0.4PDA-QHEC-PAM hydrogels for porcine blood were measured as 300, 240, and 180 seconds (s), respectively (control group: 360 seconds) (FIG. 15a and FIG. 16a). The increase in the polydopamine (PDA) content in the hydrogel induced better coagulation by reducing blood coagulation time to result in the increase in blood viscosity, due to the solubilization of interwoven microfibrils with cationic functionality and abundant adhesive PDA. Therefore, the combination of QHEC and PDA showed synergistic results in improving blood coagulation.

SEM showed platelets adhered to the hydrogel to determine the hemostasis mechanism. Platelets were adhered to all hydrogel groups (FIG. 15c). In general, the fibrous topology of natural polymer-based fibrous scaffolds exhibited high platelet adhesiveness. Therefore, it was found that the nanofiber form may play an important role in blood coagulation. Due to the availability of cationic functionality and a large amount of adhesive PDA fibrils, a significant number of platelets were attached to the surface of the 0.4PDA-QHEC-PAM hydrogel. Therefore, it was determined that the functionality with positively charge and PDA fibers could act as a hemostatic sequence that is able to promote hemostasis achieved by electrostatic interactions between the hydrogel functionality and platelets.

Hemolysis analysis of PDA-QHEC-PAM hydrogel was evaluated to determine blood compatibility. The digital image in FIG. 16b showed the color difference between the positive control (Triton-X) and the negative control (Saline). It was determined that the positive control (Triton-X) was not apparent (bright red) due to hemoglobin release from damage to the cell walls of red blood cells (RBCs). Similar to the negative control group, all hydrogel groups were observed to have a bright color that allows determination of the blood compatibility of the hydrogels. As shown in FIGS. 15b and 16b, the hemolysis rates of QHEC-PAM, 0.2PDA-QHEC-PAM, and 0.4PDA-QHEC-PAM hydrogels were calculated to be 2.5%, 1.3%, and 0.7%, respectively, indicating that all hydrogel samples were compatible with blood. Additionally, the hemolysis rate barely increased when PDA increased in the QHEC-PAM hydrogel. Hydrogels (0.2PDA-QHEC-PAM, 0.4PDA-QHEC-PAM, and Saline) showed hemolysis rates of less than 5%, demonstrating that the hemocompatible behavior of the hydrogels has wound healing properties.

### 1-10. Analysis of the Antibacterial Activity of the Hydrogel

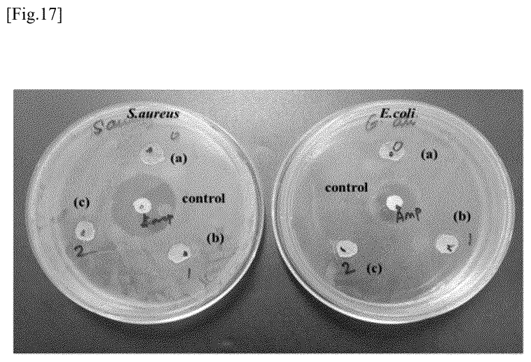

To examine that the surface of the PDA-QHEC-PAM hydrogel has a quaternary ammonium salt of QHEC and the catechol moiety of PDA exhibits favorable antibacterial activity against Gram-positive bacteria (E. coli) and Gram-negative bacteria (Staphylococcus aureus), the antibacterial activity of the PDA-QHEC-PAM hydrogel was measured via the disk diffusion method and the CFU method. The diameter of zone inhibition for all hydrogels was similar as 8 mm for E. coli and 8.6 mm for Staphylococcus aureus (FIG. 17). Additionally, a quantitative method was involved to identify the antibacterial properties of the hydrogel. The bacterial reduction rates of E. coli through treatment of QHEC-PAM, 0.2PDA-QHEC-PAM, and 0.4PDA-QHEC-PAM hydrogel were approximately 87.4%, 91.6%, and 93.3%, and the bacterial reduction rates of Staphylococcus aureus were found to be 88.6%, 91.8%, and 94.6% (FIG. 15d). From the results, it was determined that QHEC plays an essential role in bacterial reduction, and PDA delays bacterial growth when the reduction rates of the two bacteria increase slightly. Overall, the hydrogel was found to have multifunctional properties such as the tissue adhesive (FIG. 4n), antibacterial, hemostatic, and cell adhesive (FIG. 15e), with an ability of fortifying wound healing when applied as a wound dressing.

While a specific part of the present disclosure has been described in detail above, it is clear for those skilled in the art that this specific description is merely preferred example embodiments, and the scope of the present disclosure is not limited thereby. In other words, the substantial scope of the present disclosure is defined by the appended claims and their equivalents.

1. A biocompatible hydrogel (PDA-QHEC-PAM) comprising quaternized hydroxyethyl cellulose (QHEC), polydopamine (PDA), and polyacrylamide (PAM) as active ingredients.

2. The hydrogel of claim 1, wherein the hydrogel further comprises N,N′-methylenebis(acrylamide) (BIS).

3. The hydrogel of claim 2, wherein the hydrogel is in a form cross-linked by N,N′-methylenebis(acrylamide), with QHEC serving as a polymer and polydopamine and polyacrylamide serving as monomers.

4. The hydrogel of claim 1, wherein the hydrogel has one or more properties selected from the group consisting of self-adhesiveness, porosity, biodegradation, and hydrophilicity.

5. The hydrogel of claim 1, wherein the hydrogel promotes blood coagulation or water uptake.

6. The hydrogel of claim 1, wherein the hydrogel exhibits an antibacterial activity against Escherichia coli or Staphylococcus aureus.

7. A method of treating a wound or effecting hemostasis in a subject, comprising:
applying a pharmaceutical composition comprising a hydrogel (PDA-QHEC-PAM) having quaternized hydroxyethyl cellulose (QHEC), polydopamine (PDA), and polyacrylamide (PAM) as an active ingredient to a site on the subject in need thereof.

8. The method of claim 7, wherein the pharmaceutical composition is in the form of a topical preparation.

9. A method of preparing a biocompatible hydrogel (PDA-QHEC-PAM), the method comprising:
mixing quaternized hydroxyethyl cellulose (QHEC) and dopamine (DA) and solubilizing the mixture (first step); and
mixing the mixture with acrylamide (AM) and polymerizing the same (second step).

10. The method of claim 9, wherein the first step further comprises mixing ammonium persulfate (APS) with QHEC.

11. The method of claim 9, wherein the second step further comprises mixing N,N′-methylenebis(acrylamide) (BIS), ammonium persulfate (APS), and N,N,N′,N′-tetramethylethylenediamine (TEMED) with the mixture.

12. The method of claim 9, wherein the second step comprises polymerizing at 50 to 70° C. for 1 to 4 hours.
